(19) United States
(12) Patent Application Publication
(10) Pub. No.: US 2004/0097554 A1
(43) Pub. Date: May 20, 2004

(54) Heteroaryl-hexanoic acid amide derivatives as immonomodulatory agents

(75) Inventors: Matthew F. Brown, Stonington, CT (US); Anderson S. Gaweco, Stonington, CT (US); Ronald P. Gladue, Stonington, CT (US); John C. Kath, Waterford, CT (US); Christopher S. Poss, Baltic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/687,380

(22) Filed: Oct. 16, 2003

Related U.S. Application Data
(60) Provisional application No. 60/422,574, filed on Oct. 30, 2002.

(51) Int. Cl. A61K 31/4439; A61K 31/455; A61K 31/4025; A61K 31/401; C 07D 4 3/02

(52) U.S. Cl. 514/340; 514/355; 514/423; 514/422; 546/268.1; 546/315; 548/517; 548/537

(57) ABSTRACT

The present invention relates to compounds of the formula (I)

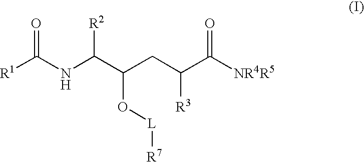

wherein R¹, R², R³, R⁴, R⁵, R⁷, and L are as described in the specification. The present invention also relates to pharmaceutical compositions that include compounds of the formula (I) and a pharmaceutically acceptable carrier. Moreover, the present invention relates to methods of using the above-described compounds and compositions to treat and prevent diseases and conditions including those that may be treated or prevented by antagonizing the CCR1 receptor.

### PRIORITY CLAIM

The present application claims priority to U.S. Patent Application Serial No. 60/422,574, filed Oct. 30, 2002, which is incorporated herein in its entirety.

### BACKGROUND OF THE INVENTION

The present invention relates to heteroaryl-hexanoic acid amide derivatives, methods of use and pharmaceutical compositions containing them.

Compounds of heteroaryl-hexanoic acid amides and their methods of manufacture are disclosed in commonly assigned U.S. patent application Ser. No. 09/380,269, filed Feb. 5, 1998, U.S. patent application Ser. No. 09/403,218, filed Jan. 18, 1999, PCT Publication No. WO98/38167, and PCT Publication No. WO99/40061, all of which are incorporated herein by reference in their entireties for all purposes.

### SUMMARY OF THE INVENTION

The present invention relates to, in one embodiment, compounds of the formula (I)

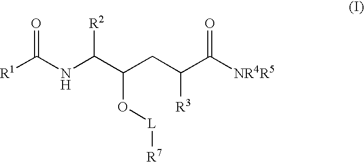

wherein R¹ is (C₂-C₉)heteroaryl optionally substituted with one or more substituents, wherein each substituent is independently hydrogen, oxygen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—, (C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

R² is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein m is zero, one, two, three or four; wherein each of said phenyl, naphthyl, (C₃-C₁₀)cycloalkyl and (C₂-C₉)heteroaryl moieties of said phenyl-(CH₂)ₘ—, naphthyl-(CH₂)ₘ—, (C₃-C₁₀)cycloalkyl-(CH₂)ₘ— and (C₂-C₉)heteroaryl-(CH₂)ₘ— groups may optionally be substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, or (C₂-C₉)heteroaryl;

R³ is hydrogen, (C₁-C₁₀)alkyl, (C₃-C₁₀)cycloalkyl-(CH₂)ₙ—, (C₂-C₉)heterocycloalkyl-(CH₂)ₙ—, (C₂-C₉)heteroaryl-(CH₂)ₙ— or aryl-(CH₂)ₙ—; wherein n is zero, one, two, three, four, five or six;

wherein the (C₁-C₁₀)alkyl moiety of said R³ (C₁-C₁₀)alkyl group may optionally be substituted with one or more substituents, wherein each substituent is independently hydrogen, halo, CN, (C₁-C₆)alkyl, (C₁-C₆)alkoxy, (C₁-C₆)alkoxy(C₁-C₆)alkyl, R⁸-L-O—, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and wherein any of the carbon-carbon single bonds of said (C₁-C₁₀)alkyl may optionally be replaced by a carbon-carbon double bond;

wherein the (C₃-C₁₀)cycloalkyl moiety of said R³ (C₃-C₁₀)cycloalkyl-(CH₂)ₙ— group may optionally be substituted by one to three substitutents, wherein each substituent is independently hydrogen, halo, CN, (C₁-C₆)alkyl, hydroxy, hydroxy(C₁-C₆)alkyl, (C₁-C₆)alkoxy, (C₁-C₆)alkoxy(C₁-C₆)alkyl, R⁸-L-O—, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

wherein the (C₂-C₉)heterocycloalkyl moiety of said R³ (C₂-C₉)heterocycloalkyl-(CH₂)ₙ— group comprises nitrogen, sulfur, oxygen, >S(═O), >SO₂ or >NR⁶, wherein said (C₂-C₉)heterocycloalkyl moiety of said (C₂-C₉)heterocycloalkyl-CH₂)ₙ— group may optionally be substituted on any of the ring carbon atoms capable of forming an additional bond with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)-, (C₁-C₆)alkyl-O—(C═O), HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

wherein the (C₂-C₉)heteroaryl moiety of said R³ (C₂-C₉)heteroaryl-(CH₂)ₙ— group comprises nitrogen, sulfur or oxygen wherein said (C₂-C₉)heteroaryl moiety of said (C₂-C₉)heteroaryl-(CH₂)ₙ— group may optionally be substituted on any of the ring carbon atoms capable of forming an additional bond with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and

wherein said aryl moiety of said R³ aryl-(CH₂)ₙ— group is optionally substituted phenyl or naphthyl, wherein said phenyl and naphthyl may optionally be substituted with from one to three substituents, wherein each substituent is independently hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

or R³ and the carbon to which it is attached form a five to seven membered carbocyclic ring, wherein any of the carbon atoms of said five membered carbocyclic ring may optionally be substituted with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; wherein one of the carbon-carbon bonds of said five to seven membered carbocyclic ring may optionally be fused to an optionally substituted phenyl ring, wherein said phenyl substitutents may be hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

Y is (C₂-C₉)heteroaryl, (C₂-C₉)heterocycloalkyl, R⁵R⁶N-sulfonyl or a group of the formula

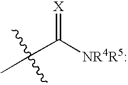

X is O, S, or NR¹²;

R⁴ is hydrogen, (C₁-C₆)alkyl, hydroxy, (C₁-C₆)alkoxy, hydroxy(C₁-C₆)alkyl, (C₁-C₆)alkoxy(C═O)—, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ—, wherein p is zero, one, two, three or four; wherein said (C₂-C₉)heterocycloalkyl, (C₂-C₉)heteroaryl, phenyl and naphthyl groups of said (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ— may be optionally substituted on any of the ring atoms capable of supporting an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

or R⁴ and R⁵ together with the nitrogen atom to which they are attached form a (C₂-C₉)heterocycloalkyl group wherein any of the ring atoms of said (C₂-C₉)heterocycloalkyl group may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—N H, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;

R⁵ is hydrogen, (C₁-C₆)alkyl or amino;

R⁶ is hydrogen, (C₁-C₆)alkyl, (C₁-C₆)alkoxy-(CH₂)₉—, (C₁-C₆)alkoxy(C═O)—(CH₂)_{g}—, (C₁-C₆)alkyl-(SO₂)—(CH₂)₉—, (C₆-C₁₀)aryloxy-(CH₂)_{g}—, (C₆-C₁₀)aryloxy(C═O)—(CH₂)_{g}—, or (C₆-C₁₀)aryl-(SO₂)—(CH₂)_{g}—, wherein g is an integer from zero to four;

R⁷ and R⁸ are each independently hydrogen, (OH)₂OP—, (OH)O₂S—, R¹¹—(NH)₂CH—(C═O)—, COOH—R¹¹—(C═O)—, R¹¹—(C₁-C₆)alkyl-(C═O)—, R¹¹—O—(C═O)—, COOH—(C═O)—, NH₂—R¹¹—(C═O)—, NH₂—R¹¹—O—(C═O)—, or R¹¹—(C═O)—;

R¹¹ is hydrogen, (C₁-C₉)alkyl, (C₂-C₉)alkenyl, (C₂-C₉)alkynyl, (C₁-C₉)alkoxy, (C₃-C₁₀)cycloalkyl, (C₂-C₉)heterocycloalkyl, (C₂-C₉)heteroaryl, aryl, (C₁-C₉)alkyl-(C═O)—(C₁-C₉)alkyl, (C₁-C₉)alkyl-(C═O)—(C₁-C₉)alkoxy, (C₁-C₉)alkoxy-(C═O)—(C₁-C₉)alkyl, (C₁-C₉)alkoxy-(C═O)—(C₁-C₉)alkoxy, (C₁-C₉)alkyl-(C═O)—(C₂-C₉)alkenyl, (C₁-C₉)alkoxy-(C═O)—(C₂-C₉)alkenyl, (C₁-C₉)alkyl-(C═O)—(C₂-C₉)alkynyl, (C₁-C₉)alkoxy-(C═O)—(C₂-C₉)alkynyl, wherein R¹¹ may be unsubstituted or substituted with one or more of hydrogen, hydroxy, carboxy, NH₂—(C═NH)—HN—, (OH)₂OP—O—, (OH)O₂S—O—, (C₁-C₉)alkyl, amino, amino(C₁-C₆)alkyl, amino(C₁-C₆)alkylamine, —NH₂—(C═O)—, thio, thio(C₁-C₆)alkyl, (C₃-C₁₀)cycloalkyl, (C₂-C₉)heterocycloalkyl, (C₂-C₉)heteroaryl, or aryl;

R¹² is hydrogen, CN, (C═O)—(C₁-C₉)alkyl, or (SO₂)—(C₁-C₉)alkyl;

L is a bond or —(CR¹³R¹⁴)—;

R¹³ and R¹⁴ are each independently hydrogen or (C₁-C₃)alkyl;

with the proviso that if L is a bond, both R⁷ and R⁸ may not be hydrogen unless R¹ is (C₂-C₉)heteroaryl substituted with one or more groups of oxygen;

with the proviso that when either R⁴ or R⁵ is hydrogen, and the other of R⁴ or R⁵ is (C₁-C₆)alkyl, R² is (C₃-C₁₀)cycloalkyl or isopropyl and R³ is (C₃-C₅)alkyl, phenyl, methylvinyl, dimethylvinyl, halovinyl, hydroxy(C₁-C₃)alkyl or amino(C₁-C₄)alkyl then R¹ must be other than indol-5-yl, 6-azaindol-2-yl, 2,3-dichloro-pyrol-5-yl, 4-hydroxyquinolin-3-yl, 2-hydroxyquinoxalin-3-yl, 6-azaindolin-3-yl, or optionally substituted indol-2 or 3-yl;

and the pharmaceutically acceptable forms of such compounds.

In preferred embodiments, the compound of formula I has the formula Ia, Ib or Ic

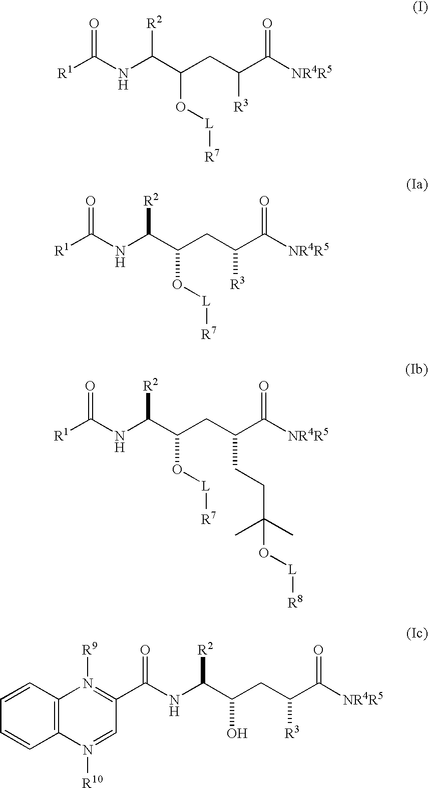

R¹, R², R⁴, R⁵, R⁷ and R⁸ are as described above; and

R⁹ and R¹⁰ are each independently oxygen or electron pairs and at least one of R⁹ and R¹⁰ are oxygen.

In another preferred embodiment, R¹ is an optionally substituted pyrazolo[3,4-b]pyridinyl, cinnolinyl, pyridinyl, 6,7-dihydro-5H-[1]pyrindinyl, benzothiazolyl, indolyl, pyrazinyl, benzoimidazolyl, benzofuranyl, benzo[b]thiophenyl, naphthalenyl, quinoxalinyl, isoquinolinyl, 5,6,7,8-tetrahydro-quinolin-3-yl or quinolinyl, more preferably pyrazolo[3,4-b]pyridin-5-yl, cinnolin-4-yl, pyridin-2-yl, 6,7-dihydro-5H-[1]pyrindin-3-yl, benzothiazol-2-yl, indol-2-yl, pyrazin-2-yl, benzoimidazol-2-yl, benzofuran-2-yl, benzo[b]thiophen-2-yl, naphthalen-2-yl, quinoxalin-2-yl, quinoxalin-6-yl, isoquinolin-1-yl, isoquinolin-3-yl, isoquinolin-4-yl, 5,6,7,8-tetrahydro-quinolin-3-yl, quinolin-2-yl, quinolin-3-yl, quinolin-4-yl or quinolin-6-yl, most preferably quinoxalin-6-yl, quinolin-2-yl, quinolin-3-yl, quinoxalin-2-yl, quinolin-4-yl or quinolin-6-yl; more preferably R¹ is an optionally substituted quinoxalin-2-yl, quinoxalin-6-yl, quinolin-2-yl, quinolin-3-yl, quinolin-4-yl or quinolin-6-yl.

In still another preferred embodiment, R² is an optionally substituted phenyl, benzyl, naphthyl, cyclohexyl, thienyl, thiazolyl, pyridyl, oxazolyl, furanyl, or thiophenyl; wherein said substituents, where the substituents are each independently hydrogen, halo, (C₁-C₆)alkyl, trifluoromethyl, trifluoromethoxy, hydroxy, —C(═O)—OH, (C₁-C₆)alkoxy, (C₁-C₆)alkoxy(C═O)—, NO₂, amino, (C₁-C₆)alkylamino, [(C₁-C₆)alkyl]₂amino, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, phenoxy, or benzyloxy; more preferably R² is optionally substituted benzyl.

In a further preferred embodiment, R³ is an optionally substituted (C₁-C₁₀)alkyl, benzyl, pyranyl or (C₃-C₁₀)cycloalkyl-(CH₂)ₙ—, wherein any of the carbon-carbon single bonds of said (C₁-C₁₀)alkyl may be optionally replaced by a carbon-carbon double bond; more preferably R³ is optionally substituted n-butyl, isobutyl, n-pentyl, 3-methyl-butyl, 2-methyl-pentyl, allyl, cyclopentyl, cyclohexyl or cycloheptyl, more preferably wherein the substituent is fluoro, (C₁-C₆)alkyl or hydroxy.

In one preferred embodiment, R⁴ or R⁵ is hydrogen, (C₁-C₆)alkyl, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, or phenyl-(CH₂)ₚ—.

In another preferred embodiment, R⁷ or R⁸ is NH₂—R¹¹—(C═O)— or R¹¹—(NH)₂CH—(C═O)— to form an amino acid ester or R⁷ or R⁸ is COOH—R¹¹—(C═O)— to form a dicarboxylic acid monoester.

Exemplary compounds of formula I include:

Phosphoric acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Sulfuric acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Phosphoric acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Sulfuric acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Phosphoric acid mono-(3(R)-carbamoyl-1(S)-{2-(3-fluoro-phenyl)-1(S)-[(quinoxaline-2-carbonyl)-amino]-ethyl}-6-methyl-6-phosphonooxy-heptyl)ester;

Sulfuric acid mono-(3(R)-carbamoyl-1(S)-{2-(3-fluoro-phenyl)-1(S)-[(quinoxaline-2-carbonyl)-amino]-ethyl}-6-methyl-6-sulfooxy-heptyl)ester;

1-Oxy-quinoxaline-2-carboxylic acid [4(R)-carbamoyl-1(S)-(3-fluoro-benzyl)-2(S),7-dihydroxy-7-methyl-octyl]-amide;

4-Oxy-quinoxaline-2-carboxylic acid [4(R)-carbamoyl-1(S)-(3-fluoro-benzyl)-2(S),7-dihydroxy-7-methyl-octyl]-amide;

1,4-Dioxy-quinoxaline-2-carboxylic acid [4(R)-carbamoyl-1(S)-(3-fluoro-benzyl)-2(S),7-dihydroxy-7-methyl-octyl]-amide;

Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S)-Amino-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S),6-Diamino-hexanoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S)-Amino-5-guanidino-pentanoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S)-Amino-3-(3H-imidazol-4-yl)-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S)-Amino-succinic acid 1-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

2(S)-Amino-pentanedioic acid 1-(3(R)-carbamoyl-1(S)-{2-(3-fluoro-phenyl)-1(S)-[(quinoxalin-2-carbonyl)-amino]-ethyl}-6-hydroxy-6-methyl-heptyl)ester;

2(S)-Amino-succinamic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2(S)-Amino-4-carbamoyl-butyric acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

3-(2,4-Dimethyl-6-phosphonooxy-phenyl)-3-methyl-butyric acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

2-Acetoxymethyl-benzoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

Succinic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Succinic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester ethyl ester;

Pentanedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Pentanedioic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester ethyl ester;

Amino-acetic acid 4(R)-carbamoyl-8-(3-fluoro-phenyl)-6(S)-hydroxy-1,1-dimethyl-7(S)-[(quinoxaline-2-carbonyl)-amino]-octyl ester;

2(S)-Amino-propionic acid 4(R)-carbamoyl-8-(3-fluoro-phenyl)-6(S)-hydroxy-1,1-dimethyl-7(S)-[(quinoxaline-2-carbonyl)-amino]-octyl ester;

2(S),6-Diamino-hexanoic acid 4(R)-carbamoyl-8-(3-fluoro-phenyl)-6(S)-hydroxy-1,1-dimethyl-7(S)-[(quinoxaline-2-carbonyl)-amino]-octyl ester;

2(S)-Amino-5-guanidino-pentanoic acid 4(R)-carbamoyl-8-(3-fluoro-phenyl)-6(S)-hydroxy-1,1-dimethyl-7-(S)-[(quinoxaline-2-carbonyl)-amino]-octyl ester;

2(S)-Amino-3-(3H-imidazol-4-yl)-propionic acid 4(R)-carbamoyl-8-(3-fluoro-phenyl)-6(S)-hydroxy-1,1-dimethyl-7(S)-[(quinoxaline-2-carbonyl)-amino]-octyl ester;

2(S)-Amino-succinic acid 1-{4(R)-carbamoyl-8-(3-fluoro-phenyl)-6(S)-hydroxy-1,1-dimethyl-7(S)-[(quinoxaline-2-carbonyl)-amino]-octyl}ester;

2(S)-Amino-pentanedioic acid 1-{4(R)-carbamoyl-8-(3-fluoro-phenyl)-6(S)-hydroxy-1,1-dimethyl-7(S)-[(quinoxaline-2-carbonyl)-amino]-octyl}ester;

Succinic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Succinic acid 4(R)-carbamoyl-8-(3-fluoro-phenyl)-6(S)-hydroxy-1,1-dimethyl-7(S)-[(quinoxaline-2-carbonyl)-amino]-octyl ester ethyl ester;

Pentanedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Pentanedioic acid 4(R)-carbamoyl-8-(3-fluoro-phenyl)-6(S)-hydroxy-1,1-dimethyl-7(S)-[(quinoxaline-2-carbonyl)-amino]-octyl ester ethyl ester;

Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester;

2(S)-Amino-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester;

Amino-acetic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester;

2(S)-Amino-propionic acid 4(R)-carbamoyl-8-(3-fluoro-phenyl)-6(S)-hydroxy-1,1-dimethyl-7(S)-[(quinoxaline-2-carb onyl)-amino]-octyloxymethyl ester;

Succinic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl)ester;

Succinic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester ethyl ester;

Pentanedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl)ester;

Pentanedioic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester ethyl ester;

Succinic acid mono-{4(R)-carbamoyl-8-(3-fluoro-phenyl)-6(S)-hydroxy-1,1-dimethyl-7(S)-[(quinoxaline-2-carbonyl)-amino]-octyloxymethyl}ester;

Succinic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester ethyl ester;

Pentanedioic acid mono-{4(R)-carbamoyl-8-(3-fluoro-phenyl)-6(S)-hydroxy-1,1-dimethyl-7(S)-[(quinoxaline-2-carbonyl)-amino]-octyloxymethyl}ester;

Pentanedioic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester ethyl ester;

(3(R)-Carbamoyl-1(S)-{2-(3-fluoro-phenyl)-1(S)-[(quinoxaline-2-carbonyl)-amino]-ethyl}-6-hydroxy-6-methyl-heptyloxycarbonyloxy)-acetic acid;

3-(3(R)-Carbamoyl-1(S)-{2-(3-fluoro-phenyl)-1(S)-[(quinoxaline-2-carbonyl)-amino]-ethyl}-6-hydroxy-6-methyl-heptyloxycarbonyloxy)-propionic acid;

Carbonic acid 2-amino-ethyl ester 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

{4(R)-Carbamoyl-8-(3-fluoro-phenyl)-6(S)-hydroxy-1,1-dimethyl-7(S)-[(quinoxaline-2-carbonyl)-amino]-octyloxycarbonyloxy}-acetic acid;

3-{4(R)-Carbamoyl-8-(3-fluoro-phenyl)-6(S)-hydroxy-1,1-dimethyl-7(S)-[(quinoxaline-2-carbonyl)-amino]-octyloxycarbonyloxy}-propionic acid;

Carbonic acid 2-amino-ethyl ester 4(R)-carbamoyl-8-(3-fluoro-phenyl)-6(S)-hydroxy-1,1-dimethyl-7(S)-[(quinoxaline-2-carbonyl)-amino]-octyl ester;

But-2-enedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

But-2-enedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Oxalic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;

Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxycarbonyloxymethyl ester;

Carbonic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester 2,3-dihydroxy-propyl ester;

Cis-but-2-enedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Oxalic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;

Trans-but-2-enedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester

Acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;

Amino-acetic acid 4(R)-carbamoyl-8-(3-fluoro-phenyl)-6(S)-hydroxy-1,1-dimethyl-7(S)-[(quinoxaline-2-carbonyl)-amino]-octyloxycarbonyloxymethyl ester; and

Carbonic acid 4(R)-carbamoyl-8-(3-fluoro-phenyl)-6(S)-hydroxy-1,1-dimethyl-7(S)-[(quinoxaline-2-carbonyl)-amino]-octyl ester 2,3-dihydroxy-propyl ester.

A second aspect of the present invention relates to pharmaceutical compositions comprising an amount of a compound of formula (I), or a pharmaceutically acceptable salt or ester thereof, and a pharmaceutically acceptable carrier.

A third aspect of the present invention relates to methods for treating or preventing a disorder or condition that can be treated or prevented by antagonizing the CCR1 receptor in a subject or inhibiting the production of metalloproteinase or cytokine at an inflammatory site in a subject, wherein the method comprises administering to a subject an effective amount of the compound of formula (I) or the composition described above.

In one preferred embodiment, the methods of the present invention are useful for treating or preventing a disorder or condition in a subject, selected from the group consisting of autoimmune diseases, acute and chronic inflammatory conditions, allergic conditions, infection associated with inflammation, viral inflammation, transplantation tissue rejection, atherosclerosis, restenosis, HIV infectivity, granulomatous diseases in a mammal, fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents; wherein the method comprises administering to a mammal a pharmaceutically effective amount of the above-described compounds or compositions.

In another preferred embodiment, the methods of the present invention are useful for treating or preventing a disorder or condition in a subject, wherein the disorder or condition is Alzheimer's disease, rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis, pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, asthma, contact dermatitis, atopic dermatitis, chronic bronchitis, chronic obstructive pulmonary disease, adult Respiratory Distress Syndrome, Respiratory Distress Syndrome of infancy, immune complex alveolitis, synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome, sarcoidosis, leprosy, tuberculosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, viral inflammation of the lung or liver, gastrointestinal inflammation, bacterial meningitis, cytomegalovirus, adenoviruses, Herpes viruses, fungal meningitis, lyme disease, and malaria; wherein the method comprises administering to a subject an effective amount of the compound of formula (I) or the composition described above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

Unless otherwise indicated, “alkyl” groups referred to herein, as well as the alkyl moieties of other groups referred to herein (e.g., alkoxy), may be linear or branched, saturated (e.g. alkanes) or unsaturated (e.g. alkenes and alkynes) and they may also be cyclic (e.g., cyclopropyl, cyclobutyl, cyclopentyl, cyclohexyl or cycloheptyl) or be linear or branched and contain cyclic moieties. Such alkyl and alkoxy groups may be optionally substituted with one, two or three halogen and/or hydroxy atoms, preferably fluorine atoms.

“Amino acid ester” shall include esters of organic acids containing at least one basic amino group and at least one carboxylic acid group. This includes the known common amino acids, such as alanine, arginine, aspartic acid, asparagine, cysteine, glutamic acid, glutamine, glycine, histidine, iso-leucine, leucine, lysine, methionine, phenylalanine, proline, serine, threonine, tryptophan, tyrosine, and valine. This also includes all other amino acids, including, but not limited to, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

“Dicarboxylic acid monoester” includes monoesters of carboxylic acid compounds containing two —COOH groups, including, but not limited to, tartronic, malic, tartaric, arabiraric, ribaric, xylaric, lyxaric, glucaric, mucic, mannaric, gluaric, allaric, altaric, idaric, talaric, glutaric, malonic, pamoic, succinic, adipic, oxalic, phthalic, sebacic, and maleic acids.

“Tricarboxylic acid monoester” includes monoesters of carboxylic acid compounds having three or more —COOH groups, including, but not limited to, citric, isocitric, citramalic, agaricic, quinic, glucuronic, glucuronolactanic, galacturonic, ascorbic, dihydroascorbic, dihydroxytartaric, and tropic acids.

Unless otherwise indicated, “halogen,” “halide,” and “halo” includes fluorine, chlorine, bromine, and iodine.

“(C₃-C₁₀)cycloalkyl” when used herein refers to cycloalkyl groups containing zero, one or two levels of unsaturation such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane, norbornanyl, and the like.

“(C₂-C₉)heterocycloalkyl” when used herein refers to pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, and the like. Said heterocycloalkyl group contains at least one carbon atom, wherein the heteroatoms may be any combination of N, O, or S. One of ordinary skill in the art will understand that the connection of said (C₂-C₉)heterocycloalkyl rings is through a carbon or a sp³ hybridized nitrogen heteroatom.

“(C₂-C₉)heteroaryl” when used herein refers to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl, benzoxazinyl, and the like. Said heteroaryl group contains at least one carbon atom, wherein the heteroatoms may be any combination of N, O, or S. One of ordinary skill in the art will understand that the connection of said (C₂-C₉)heterocycloalkyl rings is through a carbon atom or a sp³ hybridized nitrogen heteroatom.

“Aryl” when used herein refers to phenyl or naphthyl.

The symbol “−” when used between two groups of a substituent shall mean a chemical bond.

By “pharmaceutically acceptable” is meant a material that is not biologically or otherwise undesirable, i.e., the material may be administered to an individual along with the selected compound without causing any substantially undesirable biological effects or interacting in a deleterious manner with any of the other components of the pharmaceutical composition in which it is contained.

“Pharmaceutically acceptable forms” when used herein refers to any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs.

The term “subject” is meant an individual. Preferably, the subject is a mammal such as a primate, and more preferably, a human. Thus, the “subject” can include domesticated animals, livestock, and laboratory animals.

In general, “effective amount” or “effective dose” means the amount needed to achieve the desired result or results (treating or preventing the disorder or condition). One of ordinary skill in the art will recognize that the potency and, therefore, an “effective amount” can vary for the various compounds used in the invention. One skilled in the art can readily assess the potency of the compounds.

Unless otherwise noted, numerical values described and claimed herein are approximate. Variation within the values may be attributed to equipment calibration, equipment errors, purity of the materials, among other factors. Additionally, variation may be possible, while still obtaining the same result.

Compounds of the formulas I, Ia, Ib and Ic may be prepared by any method known in the art. In particular, David Fleisher, et al Advanced Drug Delivery Reviews, 1996, 19, 115-130 and Reza Oliyai Advanced Drug Delivery Reviews, 1996, 19, 275-286 teach methods of making prodrugs. Moreover, compounds of the present invention may be prepared according to the following reaction schemes and discussion. Unless otherwise indicated, the substiuents of all structural formulas in the reaction schemes and discussion that follow are the same as that defined above. Scheme 1 depicts methods of making compounds of formulas I and Ia.

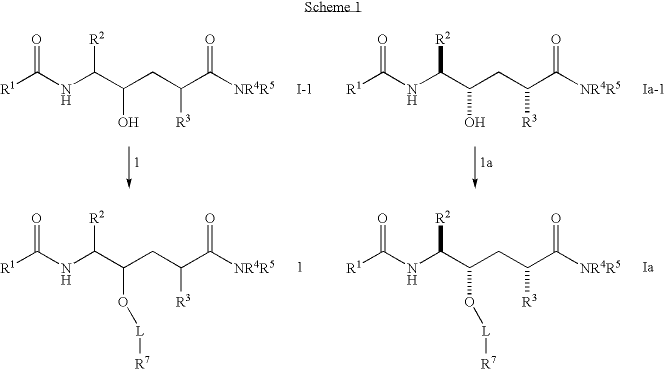

Reactions 1 and 1a of Scheme 1 depict the conversion of compounds of formulas I-1 and Ia-1 to the corresponding compounds of formulas I and Ia. These reactions fall within five general categories: conversion to the corresponding phosphates (L=a bond, R⁷═(OH)₂OP—); conversion to the corresponding sulfates (L=a bond, R⁷═(OH)O₂S—); conversion to the corresponding esters (L=a bond, R⁷═R¹¹—(NH)₂CH—(C═O)—, COOH—R¹¹—(C═O)—, R¹¹—(C₁-C₆)alkyl-(C═O)—, COOH—(C═O)—, NH₂—R¹¹—(C═O)—, R¹¹—(C═O)—); conversion to the corresponding methyleneoxy linked compounds (L=—CR¹³R¹⁴—O—, R⁷═((OH)₂OP—, (OH)O₂S—, R¹¹—(NH)₂CH—(C═O)—, COOH—R¹¹—(C═O)—, R¹¹—(C₁-C₆)alkyl-(C═O)—, R¹¹—O—(C═O)—, COOH—(C═O)—, NH₂—R¹¹—(C═O)—, NH₂—R¹¹—O—(C═O)—, or R¹¹—(C═O)—; or conversion to the corresponding carbonate linked compounds (L=bond, R⁷═R¹¹—O—(C═O)—). However, each of these conversions may be accomplished using methods well known to those skilled in the art.

For example, conversion of I-1 or Ia-1 to the corresponding phosphates (L=bond, R⁷═(OH)₂OP—) of formula I or Ia may be accomplished by reacting I-1 or Ia-1 with a dialkyl or diaryl chlorophosphate, such as diphenyl chlorophosphate or diethyl chlorophosphate, in the presence of a base, such as N,N-dimethylamino pyridine, triethylamine, or 1-methylimidazole. This reaction occurs in a polar aprotic solvent, such as methylene chloride or diethyl ether, at a temperature between 0° C. and 50° C. for a time period of 1 hour to 24 hours. The dialkyl phosphates thus formed are then converted to the phosphates of formula I or Ia. Dialkyl phosphates may be converted to phosphates by treating with an acid, such as hydrochloric acid. Diaryl or dibenzyl phosphates may be converted to phosphates by hydrogenating the compounds using standard techniques that are well known to those skilled in the art. For example, deprotection may be effected with hydrogen gas (H₂), using catalysts such as palladium on carbon (Pd/C), palladium on barium sulfate (Pd/BaSO₄), platinum on carbon (Pt/C), or tris(triphenylphosphine) rhodium chloride (Wilkinson's catalyst), in an appropriate solvent such as methanol, ethanol, tetrahydrofuran, dioxane or ethyl acetate, at a pressure from about 1 to about 5 atmospheres and a temperature from about 10° C. to about 60° C. References describing the preparation of phosphates include: J. Org. Chem. 1991, 56, 4084; Bioorg. Med. Chem. Left. 1996, 6, 1285; Tet. Left. 1988, 29, 979; Proc. Res. Dev. 2002, 6, 109.

Conversion of compounds of the formulas I-1/Ia-1 to the corresponding sulfates (L=bond, R⁷═(OH)O₂S—) of formulas I/Ia may be accomplished by reacting I-1/Ia-1 with a sulfur trioxide complex, such as trimethylamine sulfonate, pyridine sulfonate, and N,N-dimethylformamid sulfonate, either in a solvent, such as pyridine, or neat (i.e. no solvent) at a temperature between 0° C. and 50° C. for a time period of 1 hour to 48 hours. References describing the preparation of sulfates include: Org. Lett. 2000, 2, 2921; Carbohyd. Res. 2000, 329, 667; J. Am. Chem. Soc. 2000, 122, 5017; J. Chem. Soc. Perkins I 1990, 1739; Tet. Left. 1994, 35, 8795; Sulfation of Drugs and Related Compounds, Mulder, G. R., Ed.; CRC Press: Boca Raton, Fla., 1981.

Conversion of compounds of the formulas I-1/Ia-1 to the corresponding esters of formulas I/Ia (L=a bond, R⁷═R¹¹—(NH)₂CH—(C═O)—, COOH—R¹¹—(C═O)—, R¹¹—(C₁-C₆)alkyl-(C═O)—, COOH—(C═O)—, NH₂—R¹¹—(C═O)—, R¹¹—(C═O)—) may be accomplished by coupling the hydroxyl intermediates I-1/Ia-1 with the required carboxylic acid using methods well known to those skilled in the art. Such coupling reactions are generally conducted at a temperature of about −30° C. to about 80° C., preferably about 0° C. to about 25° C. Examples of suitable coupling reagents that activate the carboxylic acid functionality include: dicyclohexylcarbodiimide/hydroxybenzotriazole (DCC/HBT); dicyclohexylcarbodiimide/dimethylaminopyridine (DCC/DMAP); N-3-dimethylaminopropyl-N′-ethylcarbodiimide (EDC/HBT); 2-ethyoxy-1-ethoxycarbonyl-1,2-dihydroquinoline (EEDQ); carbonyl diimidazole (CDI); carbonyl diimidazole/dimethylaminopyridine (CDI/DMAP); and isopropyl chloroformate/triethylamine/dimethylaminopyridine (ICF/TEA/DMAP). The coupling is conducted in an inert solvent, preferably an aprotic solvent, such as acetonitirile, dichloromethane, chloroform, and dimethylformamide. One preferred solvent is dichloromethane. References describing the preparation of esters include: Helv. Chim. Acta 2000, 83, 2607; J. Org. Chem. 2000, 65, 3034; Bioorg. Med. Chem. Lett. 2001, 11, 13. The esters of formula 1/1a can also be prepared by methods well known to those skilled in the art which involve the reaction of compounds of formula 1-1/1a-1 with “activated esters” such as acid chlorides or anhydrides. A Mitsunobu coupling may be employed to prepare esters of formulas I/Ia as well, however, this approach leads to inversion of chirality of the sp³ carbon to which the hydroxyl group is attached, see: Synthesis 1981, 1.

Conversion of compounds of the formulas I-1/Ia-1 to the corresponding methyleneoxy linked analogs (L=−CR¹³R¹⁴—O—) may be accomplished by coupling the hydroxyl intermediates I-1/Ia-1 with an intermediate of the formula

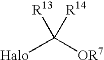

where R¹³ and R¹⁴ are as defined above and R⁷ is as defined above optionally containing protecting groups as needed to enable a successful transformation. For example, when R⁷ is a phosphate or sulfate, it may be protected as a dialkyl or diaryl phosphate or alkyl sulfate. When R⁷ is R¹¹(C═O)—, R¹¹ may contain functional groups that need to be protected for the above mentioned reaction to proceed. For example amino, carboxy, and hydroxy groups present in R¹¹ can be protected by methods well known to those skilled in the art, see also Protective Groups in Organic Synthesis, Greene, T. W., Wuts, P. G. M. John Wiley and Sons, Inc., 1991, and references cited therein. The reaction of the hydroxyl intermediates I-1/Ia-1 with the halo intermediate described above would typically be carried out in a polar aprotic solvent, such as tetrahydrofuran, methylene chloride or acetonitrile in the presence of a base, such as triethylamine, diisopropylethyamine or sodium hydride at a temperature between 0° C. and 60° C., typically ambient temperature, for a time period of 1 hour to 72 hours, typically about 12 hours.

Conversion of compounds of the formulas I-1/Ia-1 to the corresponding carbonate linked analogs (L=bond, R⁷═R¹¹—O—(C═O)—) may be accomplished by coupling the hydroxyl intermediates I-1/Ia-1 with an intermediate of the formula

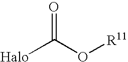

where R¹³ and R¹⁴ are as defined above and R¹¹ is as defined above. R¹¹ may contain functional groups that need to be protected for the above mentioned reaction to proceed. For example amino, carboxy, and hydroxy groups present in R¹¹ can be protected by methods well known to those skilled in the art, see also Protective Groups in Organic Synthesis, Greene, T. W., Wuts, P. G. M. John Wiley and Sons, Inc., 1991, and references cited therein. The reaction of the hydroxyl intermediates I-1/Ia-1 with the halo intermediate described above would typically be carried out in a polar aprotic solvent, such as tetrahydrofuran, methylene chloride or acetonitrile in the presence of a base, such as triethylamine or diisopropylethyamine at a temperature between 0° C. and 60° C., typically ambient temperature, for a time period of 1 hour to 72 hours, typically about 12 hours.

In Scheme 1, compounds of the formulas I-1 and Ia-1 may be prepared by any acceptable method including that described in Brown et al. (WO9838167 and references cited therein) or as shown in Scheme 1-1.

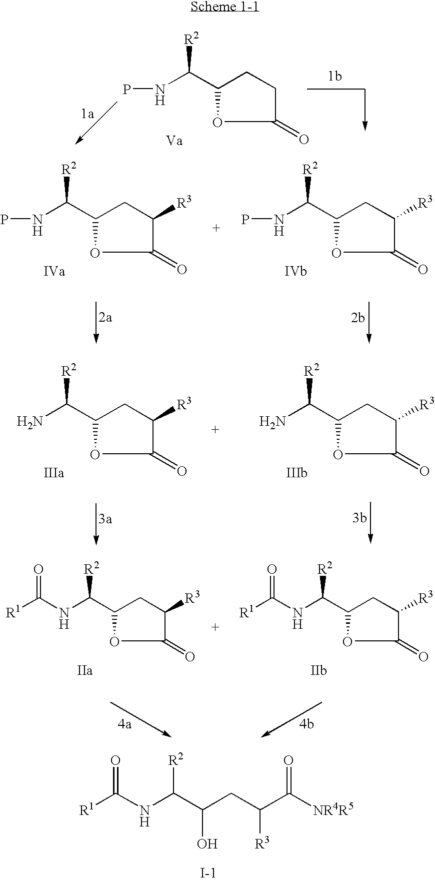

Referring to Scheme 1-1, compounds of the formula I-1, wherein either or both R⁴ or R⁵ are other than hydrogen, are prepared from compounds of the formula II (i.e. IIa and IIb) in steps 4a and 4b respectively, by reaction with a compound of the formula R⁴R⁵NH in a polar solvent at a temperature from about 0° C. to about 100° C., preferably the boiling point of the solvent used, i.e. 65° C. when methanol is the solvent. Suitable solvents include, alcohols, such as methanol, ethanol, or butanols or ethers such as glyme or dioxane (an acid catalyst is preferably used with an ether solvent). Preferably the solvent is dioxane.

Alternatively, in steps 4a and 4b compounds of formula I-1, wherein either or both R⁴ and R⁵ are hydrogen, can be prepared from compounds of formula II, (i.e. IIa and IIb) by reaction with ammonia or another volatile amine in a polar solvent at a temperature from about −10° C. to about 35° C., preferably at about 30° C. Suitable solvents include, alcohols, such as methanol, ethanol, or butanols; or ethers such as glyme or dioxane (an acid catalyst may be used with an ether solvent). Preferably the solvent is methanol.

Compounds of formula II are prepared in steps 3a and 3b of Scheme 1-1 by coupling a compound of formula III (i.e. IIIa and IIIb) with an acid of the formula R¹CO₂H. Such a coupling reaction is generally conducted at a temperature of about −30° C. to about 80° C., preferably about 0° C. to about 25° C. Examples of suitable coupling reagents which activate the carboxylic acid functionality are dicyclohexylcarbodiimide/hydroxybenzotriazole (DCC/HBT), N-3-dimethylaminopropyl-N′-ethylcarbodiimide (EDC)/HBT, 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitirile, dichloromethane, chloroform, and dimethylformamide. The preferred solvent is dichloromethane.

For a discussion of other conditions used for amide coupling see Houben-Weyl, Vol. XV, part II, E. Wunsch, Ed., George Theime Veriag, 1974, Stuttgart, and those described in M. Bodanszky. Principles of Peptide Synthesis, Springer-Verlag, Berlin (1984) and The Peptides, Analysis, Synthesis and Biology (ed. E. Gross and J. Meienhofer), Vois 1-5. (Academic Press, New York) 1979-1983.

The compounds of formula III, wherein R³ is (C₁-C₁₀)alkyl, (C₃-C₁₀)cycloalkyl-(CH₂)ₙ, (C₂-C₉)heterocycloalkyl-(CH₂)ₙ—, (C₂-C₉)heteroaryl-(CH₂)ₙ—, or aryl-(CH₂)ₙ— can be prepared by deprotection of compounds of the formula IV (i.e. IVa and IVb) as depicted in steps 2a and 2b of Scheme 1-1. Suitable protecting groups, of the formula P, include carbobenzyloxy, t-butoxy carbonyl or 9-fluorenyl-methylenoxy carbonyl.

For example:

(a) If the protecting group, P, of the compound of the formula IV is carbobenzyloxy, the latter may be removed by hydrogenation with a nobel metal catalyst such as palladium or palladium hydroxide on carbon in the presence of hydrogen. The hydrogenation is generally conducted at a temperature of about 0° C. to about 100° C., preferably about 20° C. to 50° C.

(b) If the protecting group, P, is t-butoxycarbonyl group, such group may be removed by acidolysis. Acidolysis may be conducted with HCl in dioxane or with trifluoracetic acid in methylene chloride at a temperature of about −30° C. to about 70° C., preferably about −5° C. to about 35° C.

(c) If the protecting group, P, is 9-fluorenylmethylenoxycarbonyl, such group may be removed by treatment with an amine base, preferably piperidine. This reaction may be run in piperidine as solvent at 10° C. to about 100° C., preferably at 25° C.

Compounds of the formula III, wherein R³ is substituted (C₁-C₁₀)alkyl, (C₃-C₁₀)cycloalkyl-(CH₂)ₙ or (C₂-C₉)heterocycloalkyl-(CH₂)ₙ— may be prepared from compounds of the formula IV as shown in Scheme 1-1 steps 1a and 1b, wherein R³ is (C₁-C₁₀)alkyl, (C₃-C₁₀)cycloalkyl-(CH₂)ₙ— or (C₂-C₉)heterocycloalkyl-(CH₂)ₙ—, wherein one of the carbon-carbon single bonds is replaced by a carbon-carbon double bond, by methods well known to those of ordinary skill in the art. Specifically, one example of introduction of substitution into the R³ group, a compound of formula III, wherein R³ is (C₁-C₁₀)alkyl substituted by one to three fluoro groups can be prepared from compounds of the formula IV, wherein R³ is (C₁-C₁₀)alkyl, wherein one of the carbon-carbon single bonds of said (C₁-C₁₀)alkyl has been replaced by a carbon-carbon double bond, by reaction with hydrogen fluoride in pyridine (i.e. pyridinium poly(hydrogen fluoride), in a reaction inert solvent. Suitable solvents include cyclohexane, toluene or benzene, preferably benzene. The aforesaid reaction is run at a temperature from about −78° C. to about 35° C. Preferably, this reaction is carried out in benzene at about 25° C.

Compounds of the formula IV, wherein R³ is (C₁-C₁₀)alkyl, (C₃-C₁₀)cycloalkyl-(CH₂)ₙ—, (C₂-C₉)heterocycloalkyl-(CH₂)ₙ—, (C₂-C₉)heteroaryl-(CH₂)ₙ— or aryl-(CH₂)ₙ—, wherein n is other than zero, can be prepared by reaction of a compound of formula V with a compound of the formula R³-L, wherein L is a leaving group, in the presence of a strong base in an aprotic polar solvent. Suitable leaving groups include chloro, fluoro, bromo, iodo, mesylate, triflate or tosylate. Preferably, the leaving group is a triflate, iodide or bromide. Triflates may be easily prepared according to the method of Beard, et al., J Org Chem., 38, 3673 (1973). Suitable bases include lithium dialkyl amides such as lithium N-isopropyl-N-cyclohexylamide or potassium hydride. Suitable solvents include ethers (such as THF, glyme or dioxane) benzene or toluene, preferably THF. The aforesaid reaction is conducted at about −78° C. to about 0° C., preferably at about −78° C.

Alternatively, compounds of the formula IV, wherein R³ is (C₁-C₁₀)alkyl, (C₃-C₁₀)cycloalkyl-(CH₂)ₙ— or (C₂-C₉)heterocycloalkyl-(CH₂)ₙ— can be prepared by reaction of a compound of formula V with an aldehyde or ketone precursor of R³ in an aldol condensation. For example, a compound of the formula V can be reacted with a compound of the formula R³(═O) in the presence of a base, to form an aldol intermediate of the formula

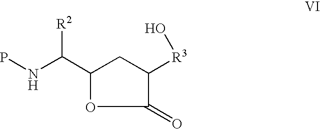

which may be isolated and taken on to final product or converted directly in the same reaction step to a compound of the formula IV by the loss of water. The degree of completion for the conversion of compounds of the formula II to the aldol product of formula I may be assessed using one or more analytical techniques, such as thin layer chromatography (tlc) or mass spectrometry. In some instances it may be possible or desirable to isolate the intermediate of formula VI. In such case, the compound of formula VI may be converted into the compound of formula IV by the elimination of water using techniques which are familiar to those skilled in the art, for example, by heating to the reflux temperature a solution of the compound of formula VI in a solvent such as benzene, toluene or xylene, in the presence of a catalytic amount of phosphorous pentoxide, benzene- or p-toluene-sulfonic acid with provision for the removal of the water generated, preferably (methoxycarbonylsulfamoyl)-triethylammonium hydroxide (Burgess reagent). Such water removal techniques may involve the use of molecular sieves or a Dean-Stark trap to isolate the water created as an azeotrope with the solvent.

The aldol reaction is typically carried out in a polar solvent such as DMSO, DMF, tetrahydrofuran (THF), methanol or ethanol, at a temperature from about −78° C. to about 80° C. Preferably, this reaction is carried out in THF at about −78° C. Suitable bases for use in the aldol formation step include potassium carbonate (K₂CO₃), sodium carbonate (Na₂CO₃), sodium hydride (NaH), sodium methoxide, potassium-tert.-butoxide, lithium diisopropylamide, pyrrolidine and piperidine. Lithium diisopropylamide is preferred. Aldol condensations are described in “Modern Synthetic Reactions,” Herbert O. House, 2d. Edition, W. A. Benjamin, Menlo Park, Calif., 629-682 (1972), J. Org. Chem., 49, 2455 (1984), and Tetrahedron, 38 (20), 3059 (1982).

Compounds of the formula IV wherein R³ is unsaturated can be converted to saturated analogues by hydrogenating the compounds containing a carbon-carbon double bond, using standard techniques that are well known to those skilled in the art. For example, reduction of the double bond may be effected with hydrogen gas (H₂), using catalysts such as palladium on carbon (Pd/C), palladium on barium sulfate (Pd/BaSO₄), platinum on carbon (Pt/C), or tris(triphenylphosphine) rhodium chloride (Wilkinson's catalyst), in an appropriate solvent such as methanol, ethanol, THF, dioxane or ethyl acetate, at a pressure from about 1 to about 5 atmospheres and a temperature from about 10° C. to about 60° C., as described in Catalytic Hydrogenation in Organic Synthesis, Paul Rylander, Academic Press Inc., San Diego, 31-63 (1979). The following conditions are preferred: Pd on carbon, methanol at 25° C. and 50 psi of hydrogen gas pressure. This method also provides for introduction of hydrogen isotopes (i.e., deuterium, tritium) by replacing ¹H₂ with ²H₂ or ³H₂ in the above procedure.

An alternative procedure employing the use of reagents such as ammonium formate and Pd/C in methanol at the reflux temperature under an inert atmosphere (e.g., nitrogen or argon gas) is also effective in reducing the carbon-carbon double bond of compounds of the formula I. Another alternative method involves selective reduction of the carbon-carbon bond. This can be accomplished using samarium and iodine or samarium iodide (SmI₂) in methanol or ethanol at about room temperature, as described by R. Yanada et. al., Synlett., 443-4 (1995).

Compounds of the formula V can be prepared by methods well known to those of ordinary skill in the art or are commercially available. Specifically, compounds of the formula Va and Vb (shown below) can be prepared by the method of Fray et al., (J. Org. Chem., 51, 4828-4833 (1986)) using an (S)-aldehyde of the formula

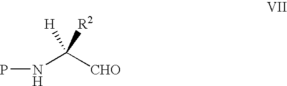

Compounds of the formula VII are prepared by reducing amino acids or amino esters to alcohols (Stanfield et al., J. Org. Chem. 46, 4799-4800 (1981), Soai et al., Bull. Chem. Soc. Jpn., 57, 2327 (1984)) followed by oxidation of the alcohols to aldehydes of the formula VII (Luly et al., J. Org. Chem., 53 (26), 6109-6112 (1988) and Denis et al., J. Org. Chem., 56 (24), 6939-6942 (1991).). Un-natural amino acids can be prepared according to the method of Myers et al., Tet. Lett. 36, (1995) and Myers et al. J. Am. Chem. Soc., 117, 8488-8489 (1995).

Alternatively, compounds of the formula V can also be made by the method of DeCamp et al., (Tetrahedron Lett., 32, 1867 (1991)).

Compounds of the formula I can be prepared according to the methods of Scheme 1, using either the minor lactone diastereomer of the formula,

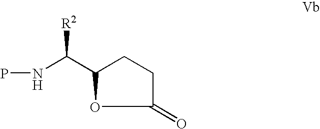

which can be prepared by the method of Fray, supra, from the (S)-aldehyde, or the alternate diastereomeric pair of the formula

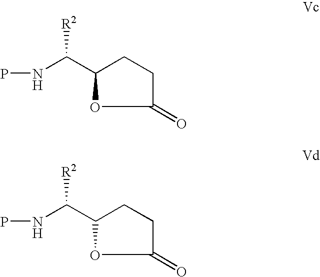

which can be prepared using the corresponding (R)-aldehyde according to the method of Fray, supra.

Aldehyde or ketone precursors of the group R³ are commercially available (e.g., cyclohexanone) or can be made by methods well known to those of ordinary skill in the art, such as described in J. Am. Chem. Soc., 90, 7001 (1968) and J. Org. Chem., 40, 574 (1975).

Scheme 2 depicts typical reactions to form compounds of formula Ib.

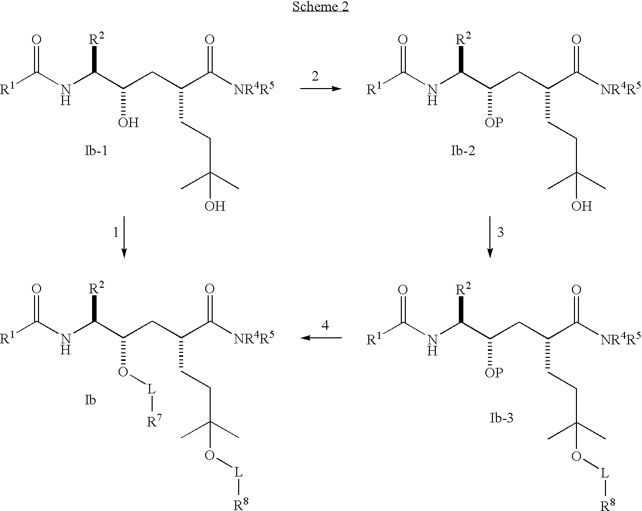

In reaction 1 of Scheme 2, compounds of the formula Ib-1 are converted to the corresponding compounds of formula Ib, wherein -L-R⁸ is —O—H, by methods described above in Scheme 1 for the conversion of I-1/Ia-1 to I/Ia. These methods fall within the five general categories of: conversion to the corresponding phosphates (L=a bond, R⁷═(OH)₂OP—); conversion to the corresponding sulfates (L=a bond, R⁷═(OH)O₂S—); conversion to the corresponding esters (L=a bond, R⁷═R¹¹—(NH)₂CH—(C═O)—, COOH—R¹¹—(C═O)—, R¹¹—(C₁-C₆)alkyl-(C═O)—, COOH—(C═O)—, NH₂—R¹¹—(C═O)—, R¹¹—(C═O)—); conversion to the corresponding methyleneoxy linked compounds (L=—CR¹³R¹⁴—O—, R⁷═(OH)₂OP—, (OH)O₂S—, R¹¹—(NH)₂CH—(C═O)—, COOH—R¹¹—(C═O)—, R¹¹—(C₁-C₆)alkyl-(C═O)—, R¹¹—O—(C═O)—, COOH—(C═O)—, NH₂—R¹¹—(C═O)—, NH₂—R¹¹—O—(C═O)—, or R¹¹—(C═O)—; or conversion to the corresponding carbonate linked compounds (L=bond, R⁷═R¹¹—O—(C═O)—), as described above.

In reaction 2 of Scheme 2, compounds of the formula Ib-1 are converted to the corresponding compounds of formula Ib-2, wherein P is a known hydroxyl protecting group, such as but not limited to, acetate, trialkylsilyl, benzyl or benzyloxycarbonyl group, by methods well known to those skilled in the art. For methods of preparing protected alcohols, see, Protective Groups in Organic Synthesis, Greene, T. W., Wuts, P. G. M. John Wiley and Sons, Inc., 1991, and references cited therein.

In reaction 3 of Scheme 2, compounds of the formula Ib-2 are converted to The corresponding compounds of the formula Ib-3 by methods described above in Scheme 1 for the conversion of I-1/Ia-1 to I/Ia. These methods fall within the five general categories of: conversion to the corresponding phosphates (L=a bond, R⁷═(OH)₂OP—); conversion to the corresponding sulfates (L=a bond, R⁷═(OH)O₂S—); conversion to the corresponding esters (L=a bond, R⁷═R¹¹—(NH)₂CH—(C═O)—, COOH—R¹¹—(C═O)—, R¹¹—(C₁-C₆)alkyl-(C═O)—, COOH—(C═O)—, NH₂—R¹¹—(C═O)—, R¹¹—(C═O)—); conversion to the corresponding methyleneoxy linked compounds (L=—CR¹³R¹⁴—O—, R⁷═(OH)₂OP—, (OH)O₂S—, R¹¹—(NH)₂CH—(C═O)—, COOH—R¹¹—(C═O)—, R¹¹—(C₁-C₆)alkyl-(C═O)—, R¹¹—O—(C═O)—, COOH—(C═O)—, NH₂—R¹¹—(C═O)—, NH₂—R¹¹—O—(C═O)—, or R¹¹—(C═O)—; or conversion to the corresponding carbonate linked compounds (L=bond, R⁷═R¹¹—O—(C═O)—), as described above.

In reaction 4 of Scheme 2, compounds of the formula V are converted to the corresponding compounds of the formula Ib by removing the hydroxyl protecting group that was installed previously in reaction 2 of Scheme 2. For methods of removing said protecting groups, see, Protective Groups in Organic Synthesis, Greene, T. W., Wuts, P. G. M. John Wiley and Sons, Inc., 1991, and references cited therein.

The compounds of formula Ib-1 may be prepared by any suitable method including the method described in Kath et al. (WO9940061) and the method depicted in Scheme 1-1 herein. Moreover, the compounds Ib-1 may be prepared from a compound of the formula V-1 as shown in Schemes 2-1,2-2, and 2-3.

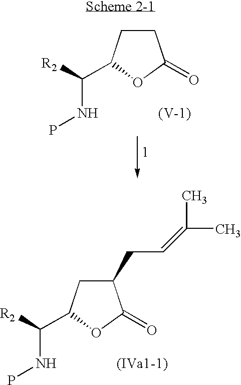

In step 1 of Scheme 2-1, the compound of the formula (IVa1-1) may be formed by reacting 4-halo-2-methyl-2-butene and a compound of the formula (v-1) in the presence of a base. Exemplary bases include lithium dialkyl amides such as lithium n-isopropyl-n-cyclohexylamide, lithium bis(trimethylsilyl)amide, lithium diisopropylamide, and potassium hydride. Suitable solvents include aprotic polar solvents such as ethers (such as tetrahydrofuran, glyme or dioxane), benzene, or toluene, preferably tetrahydrofuran. The aforesaid reaction is conducted at a temperature from about −78° C. to about 0° C., preferably at about −78° C. In one embodiment, alkylation of the lactone (v-1) is accomplished by reacting the lactone (v-1) with lithium bis(trimethylsilyl)amide and dimethylallyl bromide in tetrahydrofuran at a temperature from about −78° C. to about −50° C. Reaction times range from several hours or if an additive such as dimethyl imidazolidinone is present, the reaction may be complete in minutes.

Compounds of formula (iva1-1) may be used to produce compounds of the formula (ib-1) according to scheme 2-2:

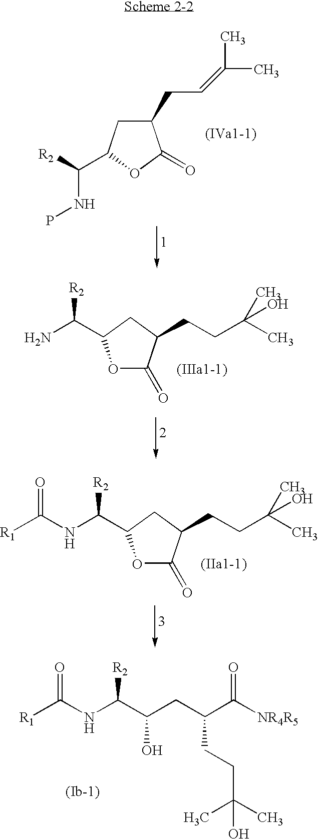

In step 1 of Scheme 2-2, a compound of the formula (IIIa1-1) is formed by reacting a compound of the formula (IVa1-1) with phosphoric acid. Preferably, this reaction occurs in any suitable solvent, such as non-alcoholic solvents. Two preferred solvents include tetrahydrofuran and dichloromethane. The reaction may take place at any suitable temperature, preferably from about −25° C. to about 120° C., more preferably from about 15° C. to about 40° C. Reaction time is dependent on temperature and batch size, amount other factors, but typically reaction time is from about 2 hours to about 14 hours.

Step 2 of Scheme 2-2 depicts coupling a compound IIIa1-1 with a compound having the formula R₁—CO—X to form a compound having the formula (IIIa1-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI), and diethylphosphorylcyanide. The coupling is conducted in an inert solvent, preferably an aprotic solvent, such as tetrahydrofuran, acetonitrile, dichloromethane, chloroform, or N,N-dimethylformamide. One preferred solvent is tetrahydrofuran. In one embodiment, quinoxaline acid is combined with CDI in anhydrous tetrahydrofuran and heated to provide the acyl imidazole. Compound IIIa1-1 is added to the acyl imidazole at room temperature to form the compound IIa1-1.

Step 3 of Scheme 2-2 includes reacting the compound of formula IIa1-1 with an amine having a formula NHR₄R₅ to form a compound of the formula (Ib-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa1-1 is dissolved in methanol which has been saturated with ammonia gas. In another embodiment, the compound IIa1-1 in methanol is treated with ammonium hydroxide in tetrahydrofuran at room temperature.

Scheme 2-3 represents an alternative method to form compounds of formula Ib-1 from compounds of formula IVa1-1.

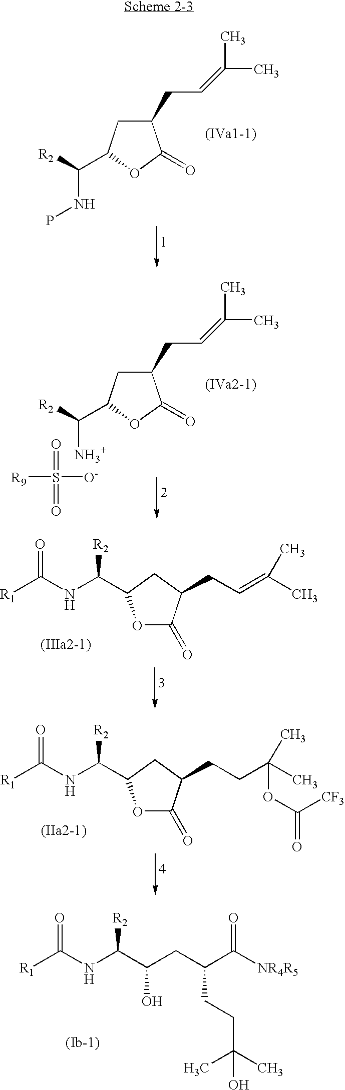

In step 1 of Scheme 2-3, a compound of the formula (IVa1-1) is reacted with a compound of the formula R₉—SO₂—OH to form a compound of the formula (IVa2-1). Any suitable acidic deprotection reaction may be performed. In one example, an excess of p-toluenesulfonic acid hydrate in ethyl acetate is introduced to the compound IVa1-1 at room temperature. Suitable solvents include ethyl acetate, alcohols, tetrahydrofuran, and mixtures thereof. The reaction may proceed at ambient or elevated temperatures. Typically, the reaction is substantially complete within two and twelve hours. The resulting compound IVa2-1 may be crystallized and separated from the reaction mixture, and may be further purified to remove impurities by recrystallization from hot ethyl acetate.

In step 2 of Scheme 2-3, the compound IVa2-1 may be coupled with a compound having the formula R₁—CO—X to form a compound of the formula (IIIa2-1). This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitrile, dichloromethane, chloroform, or N,N-dimethylformamide. One preferred solvent is methylene chloride. In one embodiment, quinoxaline acid is combined with methylene chloride, oxalyl chloride and a catalytic amount of N,N-dimethylformamide to form an acid chloride complex. The compound IVa2-1 is added to the acid chloride complex followed by triethylamine at a temperature from about 0° C. to about 25° C. to form the compound IIIa2-1.

Step 3 of Scheme 2-3 includes reacting a compound IIIa2-1 with trifluoroacetic acid to produce a compound of the formula (IIa2-1). In one embodiment, the hydration with trifluoroacetic acid occurs in methylene chloride solution at room temperature. The hydration may take several hours to complete at room temperature. A catalytic amount of sulfuric acid can be added to the reaction solution to increase the rate of reaction.

Step 4 of Scheme 2-3 includes reacting the compound of formula IIa2-1 with an amine having a formula NHR₄R₅ to form a compound of the formula (Ib-1). In one embodiment, the amine is ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa2-1 is dissolved in methanol which has been saturated with ammonia gas. In another embodiment, the compound IIa2-1 in methanol is treated with ammonium hydroxide in tetrahydrofuran at room temperature.

Scheme 3 depicts exemplary reactions to form compounds of formula Ic.

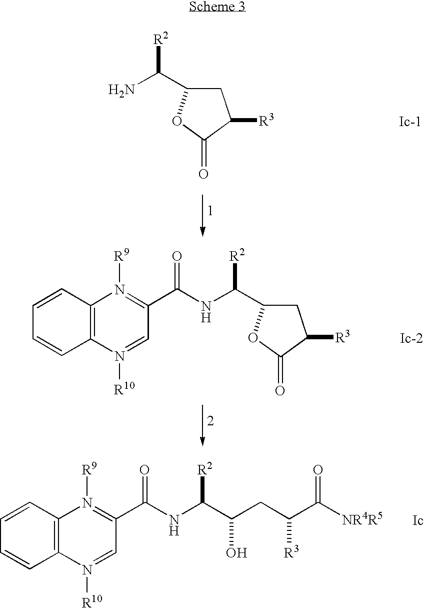

In reaction 1 of Scheme 3, compounds of the formula Ic-1 are converted to the corresponding compounds of formula Ic-2 by reacting Ic-1 with a compound of the formula VIII

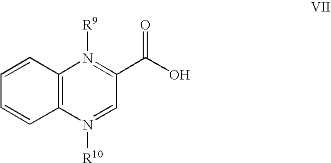

in the presence of suitable coupling reagents, such as dicyclohexylcarbodiimide/hydroxybenzotriazole (DCC/HBT); N-3-dimethylaminopropyl-N′-ethylcarbodiimide (EDC/HBT); 2-ethyoxy-1-ethoxycarbonyl-1,2-dihydroquinoline (EEDQ); carbonyl diimidazole (CDI)/dimethylaminopyridine (DMAP); and diethylphosphorylcyanide (DEPC). The coupling may be conducted in an inert solvent, preferably an aprotic solvent, such as acetonitirile, dichloromethane, chloroform, and dimethylformamide. One preferred solvent is dichloromethane. Such a coupling reaction is generally conducted at a temperature of about −30° C. to about 80° C., preferably about 0° C. to about 25° C. The compounds of formula VIII are either commercially available, or are prepared by known methods, see: J. Het. Chem. 1981, 18, 655 and J. Het. Chem. 1980, 17, 1107.

For a discussion of other conditions used for amide couplings see Houben-Weyl, Vol. XV, part II, E. Wunsch, Ed., George Theime Veriag, 1974, Stuttgart, and those described in M. Bodanszky. Principles of Peptide Synthesis, Springer-Verlag, Berlin (1984) and The Peptides, Analysis, Synthesis and Biology (ed. E. Gross and J. Meienhofer), Vois 1-5. (Academic Press, New York) 1979-1983.

In reaction 2 of Scheme 3, compounds of the formula Ic-2 are converted to the corresponding compounds of formula Ic by reacting compound Ic-2 with a compound of the formula R⁴R⁵NH in a polar solvent at a temperature from about 0° C. to about 100° C., preferably the boiling point of the solvent used, i.e. 65° C. when methanol is the solvent. Suitable solvents include, alcohols, such as methanol, ethanol, or butanols or ethers such as glyme or dioxane (an acid catalyst is preferably used with an ether solvent). Preferably the solvent is dioxane.

Alternatively, compounds of formula Ic, wherein either or both R⁴ and R⁵ are hydrogen, may be prepared from compounds of formula Ic-2 by reacting with ammonia or another volatile amine in a polar solvent at a temperature from about −10° C. to about 35° C., preferably at about 30° C. Suitable solvents include, alcohols, such as methanol, ethanol, or butanols; or ethers such as glyme or dioxane (an acid catalyst may be used with an ether solvent). Preferably the solvent is methanol.

The intermediate Ic-1 may be prepared by any suitable method including the method of Brown et al (WO9838167) or Kath et al (WO9940061) and references cited therein. Furthermore, the compound Ic-1 may be prepared as generally described in Schemes 1-1 and 2-1 for the preparation of compounds IVa and IVb (Scheme 1-1) and IVa1-1 (Scheme 2-1).

Unless indicated otherwise, the pressure of each of the above reactions is not critical. Generally, the reactions will be conducted at a pressure of about one to about three atmospheres, preferably at ambient pressure (about one atmosphere).

The compounds of the formula I, Ia, Ib or Ic which are basic in nature are capable of forming a wide variety of different salts with various inorganic and organic acids. Although such salts must be pharmaceutically acceptable for administration to animals, it is often desirable in practice to initially isolate a compound of the formula I, Ia, Ib or Ic from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

Those compounds of the formula I, Ia, Ib or Ic which are also acidic in nature, are capable of forming base salts with various pharmacologically acceptable cations. The chemical bases which are used as reagents to prepare the pharmaceutically acceptable base salts of this invention are those which form non-toxic base salts with the herein described acidic compounds of formula I, Ia, Ib or Ic. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. These salts are all prepared by conventional techniques by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Compounds of the formula I, Ia, Ib and Ic and their pharmaceutically acceptable forms (hereinafter also referred to, collectively, as “the active compounds”) are potentially useful for the treatment and prevention of a number of disorders in animals, including humans via selective antagonism of chemokines known to interact with the chemokine receptor, CCR1 (e.g., MIP-1a (CCL3), RANTES (CCL5), MCP-2 (CCL8), MCP-3 (CCL7), HCC-1 (CCL14) and HCC-2 (CCL15). The receptor, CCR1, is found on inflammatory and immunomodulatory cells (preferably leukocytes and lymphocytes) and is also sometimes referred to as the CC-CKR1 receptor. The active compounds are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic inflammatory conditions including ocular inflammation, stenosis, lung inflammation (such as chronic bronchitis, chronic obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (co-receptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention can be assessed according to procedures know to those of ordinary skill in the art. One specific example of how to determine the activity of a compound for inhibiting chemokine mediated cellular migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of >90% to be considered a valid assay.

Some compounds of the invention, although active in vivo, may not demonstrate activity in this in vitro test.

The in vivo evaluation of the compounds of the invention can be carried out by assessing their ability to inhibit cell infiltration using an air pouch model in either normal mice or mice that have been engineered to express the human CCR1 receptor. The pouch is formed on the back of the animal by subcutaneous injection of 3-4 ml of air on days 0 and 3. On the third day, animals are treated either i.p, s.c., p.o., or i.v. with the test compound then 1 ug/ml of MIP-1α is injected into the pouch at the same time and again 2 hours later. In some cases the test compound may be administered several hours prior to MIP-1α while in other cases it may be administered after the first MIP-1α injection. Further, in some cases, alternative ligands for the CCR1 receptor may be injected rather than MIP-1α (e.g. RANTES, MCP-2, MCP-3, HCC-1). Inhibition of cell infiltration is assessed by washing the pouch with sterile buffered saline and counting the cells either manually or using an automated cell counter. A compound is considered active if results show a statistically significant inhibition of cell infiltration. The compounds tested have shown an ED₅₀ result of less than about 30 μM.

The compositions of the present invention may be formulated in a conventional manner using one or more pharmaceutically acceptable carriers. Thus, the active compounds of the invention may be formulated for oral, buccal, intranasal, topical, transdermal, parenteral (e.g., intravenous, intramuscular or subcutaneous) ocular or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner. Moreover, quick dissolve tablets may be formulated for sublingual absorption.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch to provide for dry powder inhalation.

A proposed dose of the active compounds of the invention for oral, parenteral, nasal, or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents may be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, all of which are incorporated herein in their entireties for all purposes.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-Ig), BMS-224818 (CTLA4-Ig), RG-1046 (CTLA4-Ig), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

Experimental

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Melting points are uncorrected. NMR data are reported in parts per million (δ) and are referenced to the deuterium lock signal from the sample solvent (deuterochloroform unless otherwise specified). Chromatography refers to column chromatography performed using 32-63 mm silica gel and executed under nitrogen pressure (flash chromatography) conditions. Low Resolution Mass Spectra (LRMS) were recorded on either a Hewlett Packard 5989®, utilizing chemical ionization (ammonium), or a Fisons (or Micro Mass) Atmospheric Pressure Chemical Ionization (APCI) platform which uses a 50/50 mixture of acetonitrile/water with 0.1% formic acid as the ionizing agent. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Informationssysteme GmbH (ISBN 3-89536-976-4). Commercial reagents were utilized without further purification. The following abbreviations are herein used:

M is amino acid

AcOH is acetic acid

Boc is t-butoxy carbonyl

CDCl₃ is deuteriotrichloromethane

DMF is N,N-dimethylformamide

EtOAc is ethyl acetate

HCl is hydrochloric acid

HMDS is hexamethyldisilazane

IPE is isopropyl ether

MeOH is methanol

THF is tetrahydrofuran

g is grams

L is liter

M is molar

ml is milliliter

mmol is millimole

MHz is mega hertz

N is normal

psi is pounds per square inch

h is hours

min is minutes

sec is seconds

mp is melting point

RT is room temperature

Vacuo is in vacuum

˜ is roughly approximate to*

HPLC is high pressure liquid chromatography

LCMS is liquid chromatograph mass spectrometer

NMR is nuclear magnetic resonance

TLC is thin layer chromatography * Note that all numbers provided herein are approximate, but effort have been made to ensure accuracy with respect to numbers (e.g., amounts, temperature, etc.); however some errors and deviations should be accounted for.

### EXAMPLE 1

### Succinic Acid mono-(3(R)-carbamoyl-1(S)-{2-(3-fluoro-phenyl)-1-(S)-[(quinoxaline-2-carbonyl)-amino]-ethyl}-6-hydroxy-6-methyl-heptyl)ester

To a solution of quinoxaline-2-carboxylic acid [4(R)-carbamoyl-1(S)-(3-fluoro-benzyl)-2(S),7-dihydroxy-7-methyl-octyl]-amide (0.22 g, 0.45 mmol) in methylene chloride (2 mL) at 0° C. was added dimethyl aminopyridine (0.017 g, 0.14 mmol) and succinic anhydride (0.047 mg, 0.48 mmol). The reaction was warmed to ambient and stirred for 18 hours. The reaction was loaded directly onto an anion exchange column and eluted with 2 M HOAc in THF. The organics were concentrated in vacuo and the resulting oil was dissolved in methylene chloride and treated with 1 equivalent of NaOH. Trituration with diethyl ether gave the title compound as the sodium salt (0.085 g, LRMS: 583.3).

### EXAMPLE 2

### Acetic Acid 3(R)-carbamoyl-1(S)-{2-(3-fluoro-phenyl)-1(S)-[(Quinoxaline-2-carbonyl)-amino]-ethyl}-6-hydroxy-6-methyl-heptyl Ester

To a solution of quinoxaline-2-carboxylic acid [4(R)-carbamoyl-1(S)-(3-fluoro-benzyl)-2(S),7-dihydroxy-7-methyl-octyl]-amide (1.05 g, 2.07 mmol) in pyridine (4 mL) was added dimethyl aminopyridine (0.061 g, 0.50 mmol) and acetic anhydride (0.215 mL, 2.27 mmol). The reaction was stirred at ambient temperature for 2 hours. Additional acetic anhydride (0.050 mL, 0.53 mmol) was added and the reaction stirred an additional hour. The reaction was diluted with methylene chloride, and washed with 1M hydrochloric acid. The organic layer was dried over sodium sulfate, filtered and concentrated in vacuo to give the title compound ((1.26 g, LRMS: 525, 507).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of the formula (I) 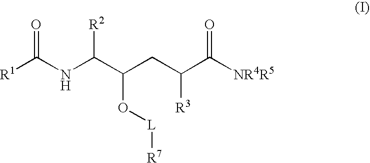
wherein R¹ is (C₂-C₉)heteroaryl optionally substituted with one or more substituents, wherein each substituent is independently hydrogen, oxygen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
R² is phenyl-(CH₂)ₘ—, naphthyl-(CH₂)ₘ—, (C₃-C₁₀)cycloalkyl-(CH₂)ₘ—, (C₁-C₆)alkyl or (C₂-C₉)heteroaryl-(CH₂)ₘ—, wherein m is zero, one, two, three or four; wherein each of said phenyl, naphthyl, (C₃-C₁₀)cycloalkyl and (C₂-C₉)heteroaryl moieties of said phenyl-(CH₂)ₘ—, naphthyl-(CH₂)ₘ—, (C₃-C₁₀)cycloalkyl-(CH₂)ₘ— and (C₂-C₉)heteroaryl-(CH₂)ₘ— groups may optionally be substituted with one or more substituents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, phenoxy, benzyloxy, (C₃-C₁₀)cycloalkyl, (C₂-C₉)heterocycloalkyl, or (C₂-C₉)heteroaryl;
R³ is hydrogen, (C₁-C₁₀)alkyl, (C₃-C₁₀)cycloalkyl-(CH₂)ₙ—, (C₂-C₉)heterocycloalkyl-(CH₂)ₙ—, (C₂-C₉)heteroaryl-(CH₂)ₙ— or aryl-(CH₂)ₙ—; wherein n is zero, one, two, three, four, five or six;
wherein the (C₁-C₁₀)alkyl moiety of said R³ (C₁-C₁₀)alkyl group may optionally be substituted with one or more substituents, wherein each substituent is independently hydrogen, halo, CN, (C₁-C₆)alkyl, (C₁-C₆)alkoxy, (C₁-C₆)alkoxy(C₁-C₆)alkyl, R⁸-L-O—, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—N H, (C₁-C₆)alkyl(C═O)—[NH](C₁-C₆)alkyl, (C₁-C₆)alkyl(C═O)—[N(C₁-C₆)alkyl](C₁-C₆)alkyl(C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and wherein any of the carbon-carbon single bonds of said (C₁-C₁₀)alkyl may optionally be replaced by a carbon-carbon double bond;
wherein the (C₃-C₁₀)cycloalkyl moiety of said R³ (C₃-C₁₀)cycloalkyl-(CH₂)ₙ— group may optionally be substituted by one to three substitutents, wherein each substituent is independently hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, R⁸-L-O—, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
wherein the (C₂-C₉)heterocycloalkyl moiety of said R³ (C₂-C₉)heterocycloalkyl-(CH₂)ₙ— group comprises nitrogen, sulfur, oxygen, >S(═O), >SO₂ or >NR⁶, wherein said (C₂-C₉)heterocycloalkyl moiety of said (C₂-C₉)heterocycloalkyl-(CH₂)ₙ— group may optionally be substituted on any of the ring carbon atoms capable of forming an additional bond with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
wherein the (C₂-C₉)heteroaryl moiety of said R³ (C₂-C₉)heteroaryl-(CH₂)ₙ— group comprises nitrogen, sulfur or oxygen wherein said (C₂-C₉)heteroaryl moiety of said (C₂-C₉)heteroaryl-(CH₂)ₙ— group may optionally be substituted on any of the ring carbon atoms capable of forming an additional bond with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; and
wherein said aryl moiety of said R³ aryl-(CH₂)ₙ— group is optionally substituted phenyl or naphthyl, wherein said phenyl and naphthyl may optionally be substituted with from one to three substituents, wherein each substituent is independently hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
or R³ and the carbon to which it is attached form a five to seven membered carbocyclic ring, wherein any of the carbon atoms of said five membered carbocyclic ring may optionally be substituted with a substituent, wherein the substituent is hydrogen, halo, CN, (C₁-C₆)alkyl, R⁸-L-O—, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl; wherein one of the carbon-carbon bonds of said five to seven membered carbocyclic ring may optionally be fused to an optionally substituted phenyl ring, wherein said phenyl substitutents may be hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
Y is (C₂-C₉)heteroaryl, (C₂-C₉)heterocycloalkyl, R⁵R⁶N-sulfonyl or a group of the formula 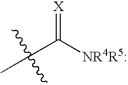
X is O, S, or NR¹²;
R⁴ is hydrogen, (C₁-C₆)alkyl, hydroxy, (C₁-C₆)alkoxy, hydroxy(C₁-C₆)alkyl, (C₁-C₆)alkoxy(C═O)—, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ—, wherein p is zero, one, two, three or four; wherein said (C₂-C₉)heterocycloalkyl, (C₂-C₉)heteroaryl, phenyl and naphthyl groups of said (C₂-C₉)heterocycloalkyl-(CH₂)ₚ—, (C₂-C₉)heteroaryl-(CH₂)ₚ—, phenyl-(CH₂)ₚ—, or naphthyl-(CH₂)ₚ— may be optionally substituted on any of the ring atoms capable of supporting an additional bond with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂ amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂-(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
or R⁴ and R⁵ together with the nitrogen atom to which they are attached form a (C₂-C₉)heterocycloalkyl group wherein any of the ring atoms of said (C₂-C₉)heterocycloalkyl group may optionally be substituted with a substituent, wherein the substituent is hydrogen, halo, CN, (C₁-C₆)alkyl, hydroxy, hydroxy-(C₁-C₆)alkyl, (C₁-C₆)alkoxy, (C₁-C₆)alkoxy(C₁-C₆)alkyl, HO—(C═O)—, (C₁-C₆)alkyl-O—(C═O)—, HO—(C═O)—(C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H(O═C)—, H(O═C)—(C₁-C₆)alkyl, (C₁-C₆)alkyl(O═C)—, (C₁-C₆)alkyl(O═C)—(C₁-C₆)alkyl, NO₂, amino, (C₁-C₆)alkylamino, [(C₁-C₆)alkyl]₂amino, amino(C₁-C₆)alkyl, (C₁-C₆)alkylamino(C₁-C₆)alkyl, [(C₁-C₆)alkyl]₂amino(C₁-C₆)alkyl, H₂N—(C═O)—, (C₁-C₆)alkyl-NH—(C═O)—, [(C₁-C₆)alkyl]₂N—(C═O)—, H₂N(C═O)—(C₁-C₆)alkyl (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[N H](C₁-C₆)alkyl, (C₁-C₆)alkyl(C═O)—[N(C₁-C₆)alkyl](C₁-C₆)alkyl, (C₁-C₆)alkyl-S—, (C₁-C₆)alkyl-(S═O)—, (C₁-C₆)alkyl-SO₂—, (C₁-C₆)alkyl-SO₂—NH—, H₂N—SO₂—, H₂N—SO₂—(C₁-C₆)alkyl, (C₁-C₆)alkylHN—SO₂—(C₁-C₆)alkyl, [(C₁-C₆)alkyl]₂N—SO₂—(C₁-C₆)alkyl, CF₃SO₃—, (C₁-C₆)alkyl-SO₃—, phenyl, (C₃-C₁₀)cycloalkyl, (C₂-C₉)heterocycloalkyl, or (C₂-C₉)heteroaryl;
R⁵ is hydrogen, (C₁-C₆)alkyl or amino;
R⁶ is hydrogen, (C₁-C₆)alkyl, (C₁-C₆)alkoxy-(CH₂)_{g}—, (C₁-C₆)alkoxy(C═O)—(CH₂)_{g}—, (C₁-C₆)alkyl-(SO₂)—(CH₂)_{g}—, (C₆-C₁₀)aryloxy-(CH₂)_{g}—, (C₆-C₁₀)aryloxy(C═O)—(CH₂)_{g}—, or (C₆-C₁₀)aryl-(SO₂)—(CH₂)_{g}—, wherein 9 is an integer from zero to four;
R⁷ and R⁸ are each independently hydrogen, (OH)₂OP—, (OH)O₂S—, R¹¹—NH)₂CH—(C═O)—, COOH—R¹¹—(C═O)—, R¹¹—(C₁-C₆)alkyl-(C═O)—, R¹¹—O—(C═O)—, COOH—(C═O)—, NH₂—R¹¹—(C═O)—, NH₂—R¹¹—O—(C═O)—, or R¹¹—(C═O)—;
R¹¹ is hydrogen, (C₁-C₉)alkyl, (C₂-C₉)alkenyl, (C₂-C₉)alkynyl, (C₁-C₉)alkoxy, (C₃-C₁₀)cycloalkyl, (C₂-C₉)heterocycloalkyl, (C₂-C₉)heteroaryl, aryl, (C₁-C₉)alkyl-(C═O)-(C₁-C₉)alkyl, (C₁-C₉)alkyl-(C═O)-(C₁-C₉)alkoxy, (C₁-C₉)alkoxy-(C═O)—(C₁-C₉)alkyl, (C₁-C₉)alkoxy-(C═O)-(C₁-C₉)alkoxy, (C₁-C₉)alkyl-(C═O)—(C₂-C₉)alkenyl, (C₁-C₉)alkoxy-(C═O)—(C₂-C₉)alkenyl, (C₁-C₉)alkyl-(C═O)—(C₂-C₉)alkynyl, (C₁-C₉)alkoxy-(C═O)—(C₂-C₉)alkynyl, wherein R¹¹ may be unsubstituted or substituted with one or more of hydrogen, hydroxy, carboxy, NH₂—(C═NH)—HN—, (OH)₂OP—O—, (OH)O₂S—O—, (C₁-C₉)alkyl, amino, amino(C₁-C₆)alkyl, amino(C₁-C₆)alkylamine, —NH₂—(C═O)—, thio, thio(C₁-C₆)alkyl, (C₃-C₁₀)cycloalkyl, (C₂-C₉)heterocycloalkyl, (C₂-C₉)heteroaryl, or aryl;
R¹² is hydrogen, CN, (C═O)-(C₁-C₉)alkyl, or (SO₂)-(C₁-C₉)alkyl;
L is a bond or —O—(CR¹³R¹⁴)—;
R¹³ and R¹⁴ are each independently hydrogen or (C₁-C₃)alkyl;
with the proviso that if L is a bond, both R⁷ and R⁸ may not be hydrogen unless R¹ is (C₂-C₉)heteroaryl substituted with one or more groups of oxygen;
with the proviso that when either R⁴ or R⁵ is hydrogen, and the other of R⁴ or R⁵ is (C₁-C₆)alkyl, R² is (C₃-C₁₀)cycloalkyl or isopropyl and R³ is (C₃-C₅)alkyl, phenyl, methylvinyl, dimethylvinyl, halovinyl, hydroxy(C₁-C₃)alkyl or amino(C₁-C₄)alkyl then R¹ must be other than indol-5-yl, 6-azaindol-2-yl, 2,3-dichloro-pyrol-5-yl, 4-hydroxyquinolin-3-yl, 2-hydroxyquinoxalin-3-yl, 6-azaindolin-3-yl, or optionally substituted indol-2 or 3-yl;
and the pharmaceutically acceptable forms of such compounds.

2. The compound according to claim 1, wherein the compound of formula (I) has the stereochemistry shown in formula (Ia): 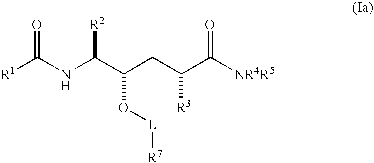
wherein R¹, R², R³, R⁴, R⁵ and R⁷ are as described in claim 1.

3. The compound according to claim 1, wherein R³ is 2-methyl-butan-2-O—R⁸ to form the compound of formula (Ib): 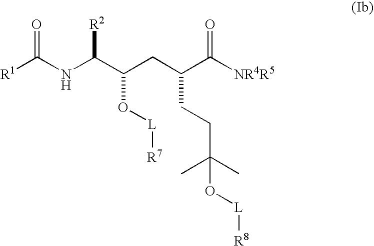
wherein R¹, R², R⁴, R⁵, R⁷ and R⁸ are as described in claim 1.

4. The compound according to claim 1, wherein R¹ is (C₂-C₉)heteroaryl substituted with one or more groups of oxygen or electron pairs of formula (Ic) 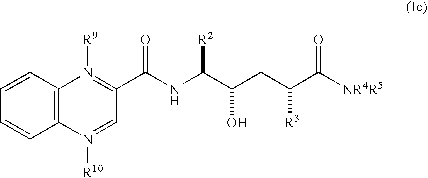
wherein R², R³, R⁴, and R⁵ are as described in claim 1; and
R⁹ and R¹⁰ are each independently oxygen or electron pairs, with the proviso that at least one of R⁹ and R¹⁰ are oxygen if R³ is (C₁-C₆)alkyl substituted with O—R⁸ and R⁸ is hydrogen and R⁷ is hydrogen.

5. The compound according to claim 1, wherein R¹ is an optionally substituted pyrazolo[3,4-b]pyridinyl, cinnolinyl, pyridinyl, 6,7-dihydro-5H-[1]pyrindinyl, benzothiazolyl, indolyl, pyrazinyl, benzoimidazolyl, benzofuranyl, benzo[b]thiophenyl, naphthalenyl, quinoxalinyl, isoquinolinyl, 5,6,7,8-tetrahydro-quinolin-3-yl or quinolinyl.

6. The compound according to claim 5, wherein R¹ is an optionally substituted quinoxalin-2-yl, quinoxalin-6-yl, quinolin-2-yl, quinolin-3-yl, quinolin-4-yl or quinolin-6-yl.

7. The compound according to claim 1, wherein R² is an optionally substituted phenyl, benzyl, naphthyl, cyclohexyl, thienyl, thiazolyl, pyridyl, oxazolyl, furanyl, or thiophenyl; wherein said substituents are each independently hydrogen, halo, (C₁-C₆)alkyl, trifluoromethyl, trifluoromethoxy, hydroxy, —C(═O)—OH, (C₁-C₆)alkoxy, (C₁-C₆)alkoxy(C═O)—, NO₂, amino, (C₁-C₆)alkylamino, [(C₁-C₆)alkyl]₂amino, (C₁-C₆)alkyl-O—(C═O)—, HO—(C═O)—(C₁-C₆)alkyl, (C₁-C₆)alkyl-O—(C═O)—(C₁-C₆)alkyl, (C₁-C₆)alkyl-(C═O)—O—, (C₁-C₆)alkyl-(C═O)—O—(C₁-C₆)alkyl, H₂N—(C═O)—, (C₁-C₆)alkyl-NH—(C═O)—[(C₁-C₆)alkyl]₂N—(C═O)—, H₂N(C═O)—(C₁-C₆)alkyl, (C₁-C₆)alkyl-HN(C═O)—(C₁-C₆)alkyl, [(C₁-C₆)alkyl]₂N—(C═O)—(C₁-C₆)alkyl, H(O═C)—NH—, (C₁-C₆)alkyl(C═O)—NH, (C₁-C₆)alkyl(C═O)—[NH](C₁-C₆)alkyl, (C₁-C₆)alkyl(C═O)—[N(C₁-C₆)alkyl](C₁-C₆)alkyl, phenoxy, or benzyloxy.

8. The compound according to claim 7, wherein R² is optionally substituted benzyl.

9. The compound according to claim 1, wherein R³ is an optionally substituted (C₁-C₁₀)alkyl, benzyl, pyranyl or (C₃-C₁₀)cycloalkyl-(CH₂)ₙ—, wherein any of the carbon-carbon single bonds of said (C₁-C₁₀)alkyl may be optionally replaced by a carbon-carbon double bond.

10. The compound according to claim 9, wherein R³ is an optionally substituted n-butyl, isobutyl, n-pentyl, 3-methyl-butyl, 2-methyl-pentyl, allyl, cyclopentyl, cyclohexyl or cycloheptyl, and the optional substituent is fluoro, (C₁-C₆)alkyl or hydroxy.

11. The compound according to claim 1, wherein R⁴ or R⁵ is hydrogen, (C₁-C₆)alkyl, (C₃-C₁₀)cycloalkyl-(CH₂)ₚ—, (C₂-C₉)heterocycloalkyl-(CH₂)ₚ—, (C₂-C₉)heteroaryl-(CH₂)ₚ—, or phenyl-(CH₂)ₚ—.

12. The compound according to claim 1, wherein R⁷ or R⁸ is NH₂—R¹¹—(C═O)— or R¹¹—(NH)₂CH—(C═O)— to form an amino acid ester or R⁷ or R⁸ is COOH—R¹¹—(C═O)— to form a dicarboxylic acid monoester.

13. The compound according to claim 1, wherein the compound is:
Phosphoric acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Sulfuric acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Phosphoric acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Sulfuric acid mono-{4(R)-carbamoyl-8-(3-fluoro-phenyl)-6(S)-hydroxy-1,1-dimethyl-7(S)-[(quinoxalin-2-carbonyl)-amino]-octyl}ester;
Phosphoric acid mono-(3(R)-carbamoyl-1(S)-{2-(3-fluoro-phenyl)-1(S)-[(quinoxaline-2-carbonyl)-amino]-ethyl}-6-methyl-6-phosphonooxy-heptyl)ester;
Sulfuric acid mono-(3(R)-carbamoyl-1(S)-{2-(3-fluoro-phenyl)-1(S)-[(quinoxaline-2-carbonyl)-amino]-ethyl}-6-methyl-6-sulfooxy-heptyl)ester;
1-Oxy-quinoxaline-2-carboxylic acid [4(R)-carbamoyl-1(S)-(3-fluoro-benzyl)-2(S),7-dihydroxy-7-methyl-octyl]-amide;
4-Oxy-quinoxaline-2-carboxylic acid [4(R)-carbamoyl-1(S)-(3-fluoro-benzyl)-2(S),7-dihydroxy-7-methyl-octyl]-amide;
1,4-Dioxy-quinoxaline-2-carboxylic acid [4(R)-carbamoyl-1(S)-(3-fluoro-benzyl)-2(S),7-dihydroxy-7-methyl-octyl]-amide;
Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S)-Amino-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S),6-Diamino-hexanoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S)-Amino-5-guanidino-pentanoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S)-Amino-3-(3H-imidazol-4-yl)-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S)-Amino-succinic acid 1-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
2(S)-Amino-pentanedioic acid 1-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
2(S)-Amino-succinamic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2(S)-Amino-4-carbamoyl-butyric acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
3-(2,4-Dimethyl-6-phosphonooxy-phenyl)-3-methyl-butyric acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
2-Acetoxymethyl-benzoic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
Succinic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Succinic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester ethyl ester;
Pentanedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Pentanedioic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester ethyl ester;
Amino-acetic acid 4(R)-carbamoyl-8-(3-fluoro-phenyl)-6(S)-hydroxy-1,1-dimethyl-7(S)-[(quinoxaline-2-carbonyl)-amino]-octyl ester;
2(S)-Amino-propionic acid 4(R)-carbamoyl-8-(3-fluoro-phenyl)-6(S)-hydroxy-1,1-dimethyl-7(S)-[(quinoxaline-2-carbonyl)-amino]-octyl ester;
2(S),6-Diamino-hexanoic acid 4(R)-carbamoyl-8-(3-fluoro-phenyl)-6(S)-hydroxy-1,1-dimethyl-7(S)-[(quinoxaline-2-carbonyl)-amino]-octyl ester;
2(S)-Amino-5-guanidino-pentanoic acid 4(R)-carbamoyl-8-(3-fluoro-phenyl)-6(S)hydroxy-1,1-dimethyl-7(S)-[(quinoxaline-2-carbonyl-amino]-octyl ester;
2(S)-Amino-3-(3H-imidazol-4-yl)-propionic acid 4(R)-carbamoyl-8-(3-fluoro-phenyl)-6(S)-hydroxy-1,1-dimethyl-7(S)-[(quinoxaline-2-carbonyl)-amino]-octyl ester;
2(S)-Amino-succinic acid {4(R)-carbamoyl-8-(3-fluoro-phenyl)-6(S)-hydroxy-1,1-dimethyl-7(S)-[(quinoxaline-2-carbonyl)-amino]-octyl}ester;
2(S)-Amino-pentanedioic acid 1-{4(R)-carbamoyl-8-(3-fluoro-phenyl)-6(S)-hydroxy-1,1-dimethyl-7(S)-[(quinoxaline-2-carbonyl)-amino]-octyl}ester;
Succinic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Succinic acid 4(R)-carbamoyl-8-(3-fluoro-phenyl)-6(S)-hydroxy-1,1-dimethyl-7(S)-[(quinoxaline-2-carbonyl)-amino]-octyl ester ethyl ester;
Pentanedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Pentanedioic acid 4(R)-carbamoyl-8-(3-fluoro-phenyl)-6(S)-hydroxy-1,1-dimethyl-7(S)-[(quinoxaline-2-carbonyl)-amino]-octyl ester ethyl ester;
Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester;
2(S)-Amino-propionic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester;
Amino-acetic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester;
2(S)-Amino-propionic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester;
Succinic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl)ester;
Succinic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester ethyl ester;
Pentanedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl)ester;
Pentanedioic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxymethyl ester ethyl ester;
Succinic acid mono-{4(R)-carbamoyl-8-(3-fluoro-phenyl)-6(S)-hydroxy-1,1-dimethyl-7(S)-[(quinoxaline-2-carbonyl)-amino]-octyloxymethyl}ester;
Succinic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester ethyl ester;
Pentanedioic acid mono-{4(R)-carbamoyl-8-(3-fluoro-phenyl)-6(S)-hydroxy-1,1-dimethyl-7(S)-[(quinoxaline-2-carbonyl)-amino]-octyloxymethyl}ester;
Pentanedioic acid 4(R)-carbamoyl-8-(3-fluoro-phenyl)-6(S)-hydroxy-1,1-dimethyl-7(S)-[(quinoxaline-2-carbonyl)-amino]-octyloxymethyl ester ethyl ester;
(3(R)-Carbamoyl-1(S)-{2-(3-fluoro-phenyl)-1(S)-[(quinoxaline-2-carbonyl)-amino]-ethyl}-6-hydroxy-6-methyl-heptyloxycarbonyloxy)-acetic acid;
3-(3(R)-Carbamoyl-1(S)-{2-(3-fluoro-phenyl)-1(S)-[(quinoxaline-2-carbonyl)-amino]-ethyl}-6-hydroxy-6-methyl-heptyloxycarbonyloxy)-propionic acid;
Carbonic acid 2-amino-ethyl ester 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
{4(R)-Carbamoyl-8-(3-fluoro-phenyl)-6(S)-hydroxy-1,1-dimethyl-7(S)-[(quinoxaline-2-carbonyl)-amino]-octyloxycarbonyloxy}-acetic acid;
3-{4(R)-Carbamoyl-8-(3-fluoro-phenyl)-6(S)-hydroxy-1,1-dimethyl-7(S)-[(quinoxaline-2-carbonyl)-amino]octyloxycarbonyloxy}-propionic acid;
Carbonic acid 2-amino-ethyl ester 4(R)-carbamoyl-8-(3-fluoro-phenyl)-6(S)-hydroxy-1,1-dimethyl-7(S)-[(quinoxaline-2-carbonyl)-amino]-octyl ester;
But-2-enedioic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Oxalic acid mono-(3(R)-carbamoyl-1(S)-{2-(3-fluoro-phenyl)-1(S)-[(quinoxaline-2-carbonyl)-amino]-ethyl}-6-hydroxy-6-methyl-heptyl)ester;
Amino-acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyloxycarbonyloxymethyl ester;
Carbonic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester 2,3-dihydroxy-propyl ester;
Cis-but-2-enedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Oxalic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Trans-but-2-enedioic acid mono-{4(R)-carbamoyl-8-(3-fluoro-phenyl)-6(S)-hydroxy-1,1-dimethyl-7(S)-[(quinoxaline-2-carbonyl)-amino]-octyl}ester;
Acetic acid 3(R)-carbamoyl-1(S)-{2-(3-fluoro-phenyl)-1(S)-[(quinoxaline-2-carbonyl)-amino]-ethyl}-6-hydroxy-6-methyl-heptyl ester;
Amino-acetic acid 4(R)-carbamoyl-8-(3-fluoro-phenyl)-6(S)-hydroxy-1,1-dimethyl-7(S)-[(quinoxaline-2-carbonyl)-amino]-octyloxycarbonyloxymethyl ester; or
Carbonic acid 4(R)-carbamoyl-8-(3-fluoro-phenyl)-6(S)-hydroxy-1,1-dimethyl-7(S)-[(quinoxaline-2-carbonyl)-amino]-octyl ester 2,3-dihydroxy-propyl ester.

14. A pharmaceutical composition comprising an amount of a compound according to claim 1, or a pharmaceutically acceptable salt or ester thereof and a pharmaceutically acceptable carrier.

15. A method for treating or preventing a disorder or condition that can be treated or prevented by antagonizing the CCR1 receptor in a subject or inhibiting the production of metalloproteinase or cytokine at an inflammatory site in a subject, wherein the method comprises administering to said subject an effective amount of the compound of claim 1.

16. A method of treating or preventing a disorder or condition selected from the group consisting of autoimmune diseases, acute and chronic inflammatory conditions, allergic conditions, infection associated with inflammation, viral inflammation, transplantation tissue rejection, atherosclerosis, restenosis, HIV infectivity, granulomatous diseases in a mammal, fibrosis, Alzheimer's disease, conditions associated with leptin production, sequelae associated with cancer, cancer metastasis, diseases or conditions related to production of cytokines at inflammatory sites, and tissue damage caused by inflammation induced by infectious agents; wherein the method comprises administering to a mammal a pharmaceutically effective amount of the compound of claim 1.

17. The method of claim 16, wherein the disorder or condition is selected from the group consisting of rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis, pulmonary fibrosis, fibrosis associated with end-stage renal disease, fibrosis caused by radiation, tubulointerstitial fibrosis, subepithelial fibrosis, scleroderma, hepatic fibrosis, primary and secondary biliary cirrhosis, asthma, contact dermatitis, atopic dermatitis, chronic bronchitis, chronic obstructive pulmonary disease, adult Respiratory Distress Syndrome, Respiratory Distress Syndrome of infancy, immune complex alveolitis, synovial inflammation caused by arthroscopy, hyperuremia, osteoarthritis, ischemia reperfusion injury, glomerulonephritis, nasal polyosis, enteritis, Behcet's disease, preeclampsia, oral lichen planus, Guillian-Barre syndrome, sarcoidosis, leprosy, tuberculosis, obesity, cachexia, anorexia, type II diabetes, hyperlipidemia and hypergonadism, sequelae associated with multiple myeloma, breast cancer, joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith, viral induced encephalomyelitis or demyelination, viral inflammation of the lung or liver, gastrointestinal inflammation, bacterial meningitis, cytomegalovirus, adenoviruses, Herpes viruses, fungal meningitis, lyme disease, and malaria.
